(19) United States
(12) Patent Application Publication
(10) Pub. No.: US 2026/0317253 A1
(43) Pub. Date: Oct. 15, 2026

(54) METHOD OF PRODUCTION OF SYNTHESIS GAS AND A REACTOR FOR PROCESSING OF SYNTHESIS GAS

(71) Applicant: Sumitomo SHI FW Energia Oy, Espoo (FI)

(72) Inventors: Eero BERG, Varkaus (FI); Rauno PEIPPO, Varkaus (FI)

(21) Appl. No.: 19/119,153

(22) PCT Filed: Oct. 17, 2023
(86) PCT No.: PCT/EP2023/078771

§ 371 Date: Apr. 8, 2025

(30) Foreign Application Priority Data
Oct. 25, 2022 (FI) 20225958

(51) Int. Cl. C07C 4/02; B01J 12/00; B01J 12/02; B01J 19/00; B01J 19/24; C10G 9/36

(52) U.S. Cl. CPC C07C 4/025; B01J 12/005; B01J 12/02; B01J 19/0013; B01J 19/244; C10G 9/36; B01J 2219/00092

(57) ABSTRACT

A method of production of synthesis gas includes providing raw gas that contains carbon compounds and sticky components to a reactor for processing of the raw gas by partial oxidation, adding oxygen and steam to the raw gas stream, reacting the oxygen and the steam with a portion of the raw gas to cause the temperature to rise to enable melting of ash and cracking of hydrocarbon compounds of sticky components, driving reactions occurring free of catalyst for cracking of sticky components by hydrogen of the steam to crack complex hydrocarbons into lighter volatile hydrocarbon fractions, controlling a feed of steam to the reactor, to decompose sticky components of the raw gas and residuals accumulate as a protective slag/dust layer on an inner wall of the reactor, and performing at least one of continuously and intermittently determining and maintaining a thickness of the protective slag/dust layer.

### CLAIM OF PRIORITY

This application is a U.S. national stage application of International Patent Application No. PCT/EP 2023/078771, filed Oct. 17, 2023, now published as International Publication No. WO 2024/088822 on May 2, 2024, which claims priority to Finnish Patent Application No. 20225958, filed Oct. 25, 2022.

### BACKGROUND OF THE INVENTION

The present invention relates to a method for production of synthesis gas. The present invention relates also to a reactor for processing of synthesis gas by partial oxidation from a raw gas containing carbon and sticky components.

### DESCRIPTION OF RELATED ART

High temperature gasification can be used to produce raw gas for power production and various chemical production plants. Many companies are developing high temperature gasification reactors to produce raw gas for further production of synthesis gas (syngas) and further liquid fuels, using feed stocks like pyrolysis oil, torrefied wood, peat, and wood dust. The aim of these processes is thus to use carbon containing feed stock to produce purified gas to a given purpose. Ash, soot, and tar are among the components that need to be purified away from the gas.

In some cases, the syngas produced by primary gasification may contain significant amounts of unreacted higher molecular weight hydrocarbons, which can be problematic for downstream equipment. One example of problematic hydrocarbons is those commonly denoted as “tars” that condense in downstream equipment, potentially causing operational and efficiency issues. These problematic hydrocarbons can be further processed by secondary gasification of the hydrocarbon-containing syngas from a primary gasifier. This configuration is similar to a primary gasifier, except that the feedstock to the secondary gasifier includes, at least in part, the crude syngas from the primary gasifier. A secondary gasifier may be used with feedstocks generated from hydrocarbon processing, such as refinery off gas (that is, crude syngas is not necessarily generated from a gasification process). In the present description, these above explained problematic hydrocarbons, tar, benzene C₆H₆, ash, and/or soot that need to be purified away from the gas are called as sticky components.

Regarding known technology, in an entrained flow gasification reactor, the ash needs to melt, whereafter the hot gas will be cooled and liquid ash will be solidified by way of a water quench or by means of a radiant cooler. Ash+Flux needs to form a protective layer on the hot face of refractory lining. It is reported that the refractory lining will be fast destroyed by the melt if the protective ash layer will not be formed or it will be washed away. On the other hand, liquid alkali metal compounds (Na and K as oxides, carbonates, hydroxides, chlorides, etc.) are very aggressive and corrosive in high temperatures. However, if the ash in a conventional high temperature gasification reactor will not become fully liquid, the ash will block the reactor. Still, tar removal is needed in fluidized bed gasification processes when aiming at very clean gas for various synthesis applications. Thermal cracking or tar removal is a known technology itself. In thermal cracking temperatures exceed 1000 °C =>ash is melting on the heat transfer surfaces

U.S. Pat. No. 7,587,995B2 , shows a synthesis gas cooler for extracting heat from synthesis gas produced by a gasification process. The synthesis gas cooler comprises a shell having a synthesis gas inlet and a synthesis gas outlet; a fluid-cooled flue contained within the shell for receiving the synthesis gas fluid-cooled radiant heat transfer surface is partially extending within the flue for cooling the synthesis gas, and means for conveying the synthesis gas from the outer flue to the outlet. The main objective in the disclosure is to transfer heat from the synthesis gas so that the ash has a reduced tendency to stick to the cooling tubes and to cause deposition and plugging. The document explains that the heat transfer surface design should be based on achieving the required absorption without the use of soot blowers to clean the heat transfer surface of deposits, which will accumulate during operation. The document explains that, based upon experience at current gasification units, an equilibrium fouling and absorption rate is achieved over time. It is noted that such equilibrium conditions are also attained in industrial and utility boilers firing similar fuels, and the performance of such boilers is quite manageable. Experiences indicate that due to slag buildup on sootblowers during service, penetration of corrosive gases, and breakdown of seal systems in-gas stream, removable sootblowers are unacceptable from a practical maintenance and availability standpoint. The document discloses cooled hanging partition walls within a gas cooler enclosure to maintain desired cooling of gas without cleaning the partition walls by soot blowers.

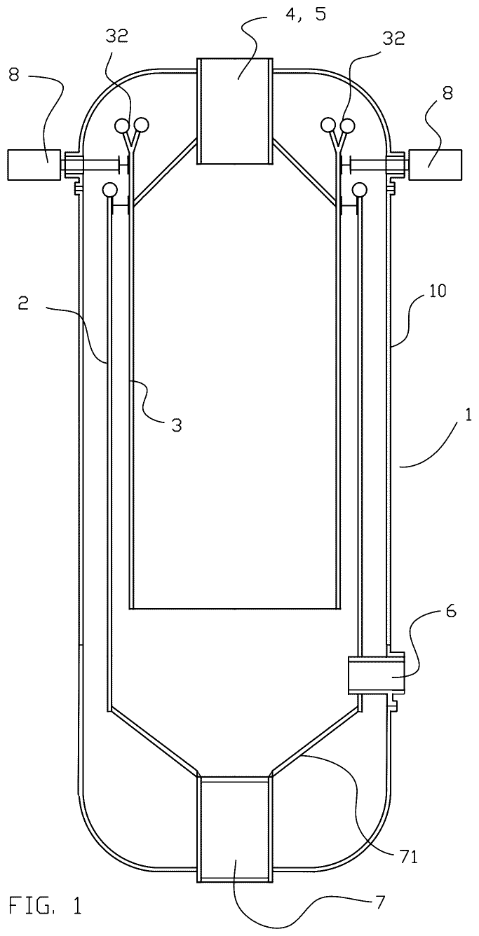

International Patent Document WO2012085345 A1 discloses (paragraph [0040], claims 1, 9, FIG. 1) a method of gasifying solid fuel in a gasification reactor (12, 12′), where the product gas is partially oxidized and its temperature is increased, whereby thermal cracking of the components of the product gas is achieved. This document discloses that, when the gas is cooled down with a radiation heat exchange cooler (41), softened and/or melt fly ash sticks to a certain extent also to the walls of the lower portion of the gas treatment reactor (20) and solidifies to the surface thereof. For this purpose, rapping hammer type soot blowers (44) are preferably provided in connection with the walls of the lower portion of the gas treatment reactor, by means of which material solidified and accumulated on the walls can be removed.

U.S. Pat. No. 7,037,473 B1 discloses (abstract, column 3, lines 15 to 55, claim 1) a gasification reactor for the gasification of carbon-and ash-containing fuel, residual and waste materials using an oxygen-containing oxidizing agent. For the thermal protection of the reactor, there is a cooling gap (5) filled with water. On the inside, the cooling gap (5) is delimited by a cooling wall (4). When ash-containing fuel is used, liquid slag which forms in the reactionchamber (1), is cooled on the cold surface of the cooling wall (4). Its protective layer solidifes and forms a refractory lining as a layer of slag which grows toward the reaction chamber 1 until the temperature has reached the melting point of the slag. The further slag, which is then ejected, runs of as a film of slag and is discharged together with the hot crude gas via the opening (8).

U.S. Patent Application Publication No. 2010143216A1 discloses (WPI abstract, paragraph [0034]) a reactor vessel for preparing syngas having a chamber linked to a diptube via a slap tap that has a tubular part connected to an opening of a frusto-conical part of chamber, where half of vertical length of tubular part extends below discharge opening. In this patent, the frusto-conical part predicts blockage by the slag by measuring the temperature of the used cooling water or steam make in conduits of the frusto-conical part, and indicates a decrease in temperature of the used cooling water or a decrease in steam for a growing layer of the slag. Thus, operating the reactor vessel closer the optimal gasification temperature while simultaneously monitoring the slay layer thickness to minimize blocking risk by the slag is disclosed.

An object of the invention is to provide a method for production of synthesis gas in which the performance is considerably improved compared to the prior art solutions.

Another object of the invention is to provide a method wherein residual substances and particles can be removed from a raw gas easily and effectively.

An object of the invention is to provide a reactor for processing of synthesis gas by partial oxidation from a raw gas containing carbon and sticky components.

### SUMMARY OF THE INVENTION

Objects of the invention can be met substantially as is disclosed in the independent claims and in the other claims describing more details of different embodiments of the invention.

An embodiment of the present invention is a method for production of synthesis gas, the method comprising the following steps: providing raw gas that contains carbon compounds and sticky components to a reactor for processing of the raw gas by partial oxidation, adding oxygen and steam to the raw gas stream, reacting the oxygen and the steam with a portion of the raw gas to cause the temperature to rise to enable melting of ash and cracking of hydrocarbon compounds of the sticky components, driving reactions occurring free of catalyst for cracking of sticky components by hydrogen of the steam to crack complex hydrocarbons into lighter volatile hydrocarbon fractions, controlling a feed of steam to the reactor, to decompose sticky components of the raw gas and residuals accumulate as a protective slag/dust layer on an inner wall of the reactor, the reactor comprising a temperature-controlled inner wall and an outer wall, being arranged in a coaxial configuration with respect to each other, the inner wall comprising a number of water tube panel walls on a suspended support and the outer walls being fixed wall, and performing at least one of continuously and intermittently determining and maintaining a thickness of the protective slag/dust layer such that, when the slag/dust layer thickness has accumulated to a predetermined value, the slag/dust layer is dropped to the bottom of the reactor by rapping at least one of the inner panel walls at a time.

Another embodiment of the invention is a reactor for processing of synthesis gas by partial oxidation from a raw gas containing carbon and sticky components, the reactor comprising: a first intake passage for feeding the raw gas;
at least one second intake passage for controllably feeding steam and oxygen such that oxygen and steam are capable of reacting with a portion of the raw gas to cause the temperature to rise to enable melting of ash and cracking of hydrocarbon compounds of the sticky components, the reactions occurring free of catalyst for cracking of sticky components being capable of being driven by hydrogen of the steam to crack complex hydrocarbons into lighter volatile hydrocarbon fractions, an exhaust passage for exit of synthesis gas; a second exhaust passage for removing at least one of separated condensed and separated solidified material; an outer wall in a fixed configuration defining an internal space; an inner wall assembly configured on a suspended support inside the outer wall, the inner wall being formed of a number of temperature-controlled water tube panel walls; the outer wall and the inner wall assembly being arranged in coaxial configuration in relation to each other and a longitudinal axis of the reactor; and at least one rapping device arranged at an upper part of the inner wall for removal of a slag/dust layer on the inner wall.

Thus, the reactor utilizes the method to treat raw gas that contains carbon compounds and sticky components for processing of the raw gas by partial oxidation, wherein oxygen and steam are added to the raw gas stream. In partial oxidation, oxygen and steam react with a portion of the raw gas to cause the temperature to rise, to enable melting of ash and cracking of hydrocarbon compounds of the sticky components. Depending on the raw gas composition, the temperature should be in a range of 1100 to 1400 °C to melt ash and crack tar. Here, a gasifier upstream of the reactor may be operated at a regular lower temperature to avoid issues with alkali and other corrosive components. As a consequence, the raw gas produced in the gasifier and entering the reactor contain sticky components, such as tar. As the raw gas is being led to the reactor, added O₂/steam reacts with a portion of the gas to cause the temperature to rise to about 1100 to 1400 °C, then, ash melts and tar cracks. The reactions occurring free of catalyst responsible for cracking are driven by hydrogen (from steam) to crack complex hydrocarbons into lighter (volatile) hydrocarbon fractions: C_{w}H_{z}+O₂+steam→CₓH_{y}+H₂Oᵥ, where w and z are much greater than x and y. Free of catalyst means that there are no additional substances included or involved as a catalyst in the reaction. After the reaction, some residual substances and particles still remaining will be captured by the temperature-controlled inner wall of the reactor.

The aim of the present process is to maintain a controlled accumulation of residuals forming a protective slag/dust layer on the reactor walls, in the temperature range where the tars of the raw gas will be decomposed. A too thick slag/dust layer causes plugging of the temperature-controlled walls and thus preventing controllability of the process. Plugging of the reactor is prevented by rapping the inner walls that captures the slag/dust. However, formation of the slag/dust layer is essential to the process, otherwise, raw gas purification does not happen and gas separation following the reactor will deteriorate. The accumulation is targeted, especially on the inner wall assembly configured on suspended support inside the outer wall and the inner wall being formed of a number of temperature-controlled water tube panel walls. Here, such a single hanging sector, section, or panel of the inner wall is called as an inner panel wall.

The temperature of surfaces where slag/dust is to be accumulated during operation should be the following:
no less than 200 °C to avoid any condensation of remaining traces of tar; and
no more than 350 °C to allow effective condensation of alkali and keep corrosion at bay.

Temperature in the reactor may advantageously be controlled so that the reactor has a low corrosion rate below one millimeter per annum when exposed to any liquid alkali metal, sulphur or chlorine compounds, while the crack of hydrocarbon compounds of the sticky components is still the primary factor for process temperature within the reactor. The first ash will deposit onto the inner wall as a solid, and, as the deposit will grow, its surface will be increasingly softer as its temperature will be closer to the gas temperature. The deposit growth will be interrupted by rapping the inner panel walls in a certain time interval, requiring collection of the deposit from the bottom of the reactor. The thickness of the protective slag/dust layer is being continuously and/or intermittently determined and maintained such that, when the slag/dust layer thickness has accumulated to a predetermined value, the slag/dust layer is dropped to the bottom of the reactor by rapping at least one of the inner panel walls at a time. The cleaning action of the hammer rely on the fact that the slag/ash in direct contact with the panel surface (200-350 °C) will be solid. It can be rapped off and down to the bottom of the reactor/hopper only at the boundary layer between deposit and inner wall when the boundary layer temperature is cooled enough. Rapping breaks the bond and drops the deposits in lumps/sheets, which are bulky enough not to have time to melt again while falling down to the hopper. Immediately after the rapping, a new slag/dust layer starts to accumulate on the surfaces of the inner wall.

According to an embodiment, the rapping device is a spring hammer. When considering the cleaning up of the heat transfer surfaces, i.e., the inner wall, the spring hammer based technology has found to be the most efficient one in this solution. In this invention, the inner wall comprising a number of inner panel walls are of a hanging type and cleaned by spring hammers. This is very efficient. Also, in operation, if needed, the changing of hanging inner walls is easy and not very time consuming. Naturally, in maintenance operations, the most time-consuming part is a cooling down procedure of the reactor so that the reactor or, possibly, its enclosure, heat shield, pressure shell, or the like, can be safely opened.

One objective of the present invention is to produce synthesis gas that fits easily to the various following processes. If the gasification temperature in the reactor is under a melting temperature of the ash, but over 1100 °C, the ash will become soft or becoming hemispherical state. If the chemical composition of ash is on the area of more alkaline, the ash won't totally melt in the reactor when the gasification temperature is under 1400 °C. According to the experiments on the invention, the gasification temperature range of 1100 °C-1300 °C is suitable for producing low tar containing gas. That brings one desired property, the produced synthesis gas by the method can be cooled down to +5 °C-+20 °C without problems. Cooling of the synthesis gas to low temperatures is typically needed because, following the water wash for removing ammonium, chlorides, etc., and because of possible compression. Chlorine removal may be needed for further processing and to protect shift-conversion catalyst. After shift conversion the most widely used process is so called a Rectisol-process. This physical wash will remove CO₂ and sour gases from synthesis gas quite well. It is commonly known that Rectisol-process is a physical wash that may handle many hydrocarbons (ex. methane) and, i.e., a small amount of tars in the gas are no problem in the Rectisol-process.

According to an embodiment of the invention the thickness of the protecting layer of the slag/dust on inner walls of the reactor is controlled by rapping the inner walls in sequence so that the temperature of the water/steam inside the inner walls is maintained within desired range. Thus, the thickness of slag/dust layer is determined by calculation based on temperature difference between ingoing and outcoming water/steam of the inner panel walls.

According to an embodiment of the invention, the slag/dust layer is dropped to the bottom of the reactor by rapping each one of the inner panel walls at a predetermined time interval. The intention for this action is that, when the slag/dust layer is dropped to the bottom of the reactor by rapping, only one or a part of the inner panel walls at a predetermined time interval, the accumulation in other inner panel walls is still in another phase of the cycle. For illustrative example, if the accumulation time for a fully grown slag/dust layer is one hour and there are six separate inner panel walls, it gives a theoretical interval of ten minutes to rap one inner panel wall while the other inner panel walls are at a different state of accumulation. This makes the process much more stable when not all the inner panel walls are rapped at the same time.

By controlling an insulating thickness of the slag/dust layer on the inner wall, the cracking of sticky components in the reactor is kept in balance. Also, part of possible alkali metal, sulphur, and/or chlorine components are being removed from the process as attached to the slag/dust. The accumulation of the slag/dust layer is not necessarily linear, but can as well be logarithmic or exponential, depending on the process parameters. Therefore, it is advisable to have different tools to decide on the time interval for rapping the inner walls. The controlling of the insulating thickness or value or mass is one of those possible tools.

The explanation above provides for a method for production of synthesis gas which performance is considerably improved.

Regarding the reactor construction, the outer wall is in a fixed configuration defining an internal space. According to an embodiment, also the outer wall is formed of a number of temperature-controlled water tube panel walls. Preferably, the outer wall is being formed of 6, 7, 8, 9, or more water tube panel walls having a substantially planar cross section and joined together to form a continuous array of panels. Preferably, the outer wall is gas tight.

The inner wall assembly is configured on a suspended support inside the outer wall and the inner wall being formed of a number of temperature-controlled water tube panel walls, here are referred to as inner panel walls. Preferably, the inner wall is being formed of 6, 7, 8, 9, or more inner panel walls having a substantially planar cross section. Each inner panel wall is hanging on its own suspended support and thus forming an array of inner panel walls. According to an embodiment, the inner wall has the same number of inner panel walls as does the outer wall.

According to an embodiment, the inner wall and the outer wall are temperature controllable by means of fluid temperature inside the inner and the outer walls. Water and steam are among the most suitable fluids for controlling the temperature of the inner walls and the outer walls. Those water/steam-based control systems are well-known in the industry, so there is less effort needed to adapt an existing control system for this purpose.

According to an embodiment, the reactor is inside an enclosure that is formed as a pressure shell. However, if the pressure inside the reactor is designed to be relatively low, for example, less than 200 kPa, the outer wall as such may be sufficient to form the closed space inside the reactor. According to an embodiment, the outer wall inside the enclosure is in a fixed configuration defining an internal space and the outer wall being formed of a number of temperature-controlled water tube panel walls, the enclosure surrounds outside the outer wall. These form a coaxial configuration of the reactor. The inner wall forms the innermost array of panels. The outer wall is in the middle and the enclosure forms the outermost layer. In an embodiment, the coaxial configuration is such that a normal of the inner wall is substantially perpendicular to an imaginary vertical axis of the reactor. Naturally, the reactor should be heat insulated in one way or another so that it would be feasible to have those operational temperatures inside the reactor without significant heat losses. There may be a heat insulated enclosure, a heat shield construction, a pressure shell, or the like.

According to an embodiment, the inner wall is shorter than the outer tube panel wall. This has the effect that the inner panels are easy to rap and there is enough room below the inner wall for the slag/dust layer to be dropped.

The exemplary embodiments of the invention presented in this patent application are not to be interpreted to pose limitations to the applicability of the appended claims. The verb “to comprise” is used in this patent application as an open limitation that does not exclude the existence of also unrecited features. The features recited in depending claims are mutually freely combinable unless otherwise explicitly stated. The novel features that are considered to be characteristic of the invention are set forth in particular in the appended claims.

### BRIEF DESCRIPTION OF THE DRAWINGS

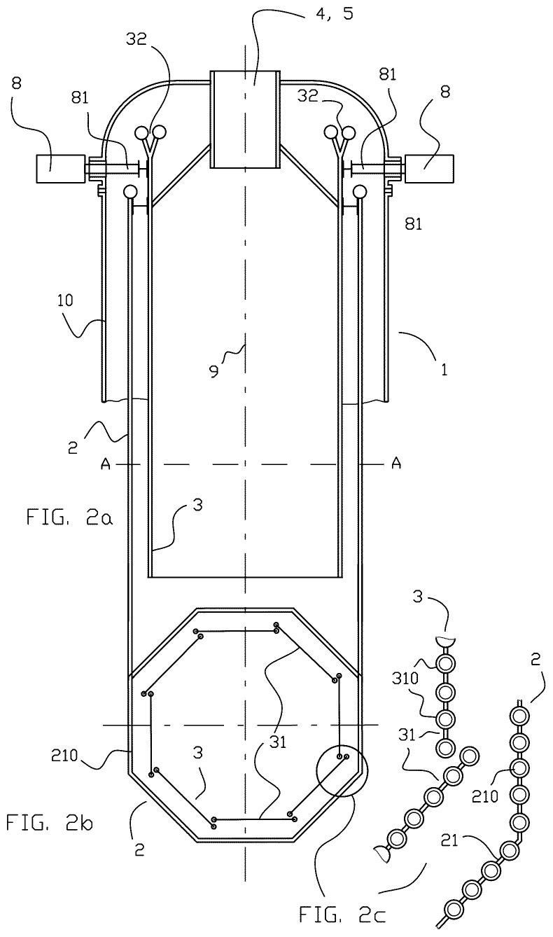

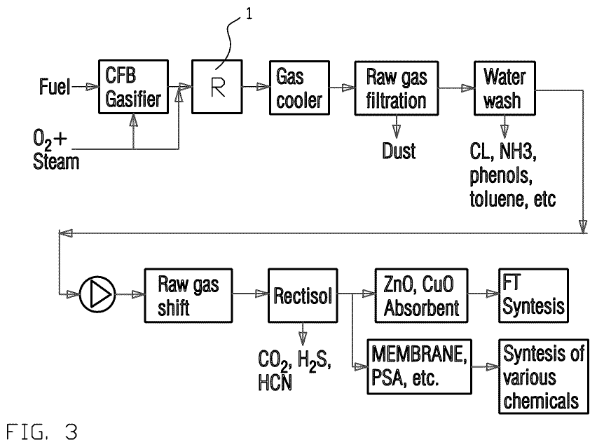

In the following, the invention will be described with reference to the accompanying exemplary, schematic drawings, in which FIG. 1 illustrates a reactor according to an embodiment of the invention, FIGS. 2a, 2b, and 2c illustrate some details of the reactor according to another embodiment of the invention, FIG. 3 illustrates an application of the present invention in a larger context of synthesis gas production.

### DETAILED DESCRIPTION OF THE INVENTION

FIG. 1 schematically depicts a reactor 1 for processing of synthesis gas by partial oxidation from a raw gas containing carbon and sticky components. The reactor comprising: an enclosure 10 formed as a shell, a first intake passage 4 for feeding the raw gas, at least one second intake passage 5 for feeding steam and oxygen, here the first 4 and the second intake passages 5 being combined to one intake passage 4, 5, an exhaust passage 6 for exit of synthesis gas, a second exhaust passage 7 for removing separated condensed and/or solidified material, an outer wall 2 inside the enclosure 10 in a fixed configuration defining an internal space, an inner wall 3 assembly configured on suspended support 32 inside the outer wall 2, at least one rapping device 8 is arranged at an upper part of the inner wall 3 for removal of a slag/dust layer on the inner wall 3, and a hopper 71 forming a base where the removed slag/dust layer may drop when the inner wall 3 is being rapped. The hopper 71 has an inclined surface that leads the condensed and/or solidified material to the proximity of the second exhaust passage 7. Preferably, the second exhaust passage 7 has a gas-tight dual hatch arrangement (not shown in the figures) that enables collecting the condensed and/or solidified material during continuous operation of the reactor 1. In that kind of dual hatch arrangement, one of the hatches is always closed, meaning that both hatches are not open simultaneously during operation, so there is no direct connection from inside the reactor 1 to the atmosphere.

FIG. 2a depicts a schematic side view of a reactor 1 for processing of synthesis gas by partial oxidation from a raw gas containing carbon and sticky components. FIG. 2b depicts a cross section of the reactor of FIG. 1 taken at A-A and FIG. 2c depicts an enlarged view of encircled detail of FIG. 2b. The reactor 1 comprises an enclosure 10 formed as a shell, depending on the process conditions as explained above, the enclosure 10 may formed as a pressure shell or as a thermal insulation, a first intake passage 4 for feeding the raw gas, at least one second intake passage 5 for feeding steam and oxygen, an outer wall 2 inside the enclosure 10 in a fixed configuration defining an internal space and the outer wall 2 being formed of a number of temperature-controlled water tube panels forming the outer panel walls 21, the panels may preferably be arranged as a continuous array as depicted in FIG. 2b and FIG. 2c, an inner wall 3 assembly configured on suspended support 32 inside the outer wall 2 and the inner wall 3 being formed of a number of separate temperature-controlled water tube inner panel walls 31, the suspended support 32 enables the inner panel walls 31 to vibrate or swing when rapped, the outer wall 2 and the inner wall 3 assembly are arranged in coaxial configuration in relation to each other and a longitudinal axis 9 of the reactor 1 as depicted in cross-sectional view of FIG. 2b, at least one rapping device 8 is arranged at an upper part of the inner wall 3 for removal of a slag/dust layer on the inner wall 3.

According to an embodiment of the invention, the interior of the reactor is made of water/steam-cooled tube wall panels according to the exemplar embodiments shown on FIGS. 2a, 2b, and 2c. The inner wall and outer wall may comprise, e.g., twelve separate sections of wall panels, here called as outer panel wall 21 and inner panel wall, 31. The width of each panel section may be, for example, 0.6 m and a height of 5 m. Each panel section will be separately cleanable by spring hammer rapping. The inner panel walls 31 forming the inner wall 3 are fast and easily replaceable, as these can be formed as modular spare parts. Preferably the construction is made of water tubes 310, 210 having a welded fin in between each water tube 310 to 310 or 210 to 210. The inner wall 3 may be formed of 6, 7, 8, 9, or more inner panel walls 31 having a substantially planar cross section. Similarly, the outer wall 2 is formed of 6, 7, 8, 9, or more outer panel walls 21 having a substantially planar cross section. Preferably, the inner wall 3 has the same number of panels 31 as the outer wall 2, so that the spacing between the inner wall and the outer wall remains about the same or equal around the circumference of the inner wall 3/outer wall 2.

The reactor 1 may be dimensioned according to the following example. (Raw gas flow 45 Nm³/s (combustion heat of raw gas before the reactor 450-500 MW). Gas pressure 25 bar (2500 kPa). Temperature in the reactor 1300 °C. Cooling water/steam temperature is 250 °C. Gas velocity in the reactor 2 m/s. Gas retention time in the reactor 2 s. The total temperature controlled wall area is 40 m². Area of the roof is 5 m². The area of one water tube panel wall section is 5.5 m².

If oxygen+steam will be fed to the reactor in a relation to the raw gas so that the temperature will be 1200-1300 °C in the reactor, the ash will become sticky and will accumulate on the wall surfaces of the reactor. The ash deposit grows or accumulates on the walls to about 100 mm thick. That would be one possible start for a sequenced of cleaning of wall panel sections one by one. The panels would be freely suspended from top to be efficiently rapped. The inside surface of the inner wall 3 can be made rough, for example, by overlay welding (which at the same time provides additional corrosion/erosion protection). According to testing experiences after cleaning by spring hammer, there can be left up to 5 mm thin residual ash layer on a cooled water tube panel wall.

FIG. 3 shows an exemplary embodiment of the present reactor 1 and method as claimed herein as a part of a larger production chain of synthesis gas. Upstream of the reactor is a gasifier delivering the raw gas. Then, there are sources for oxygen and steam. These are combined in the reactor as explained here earlier. Downstream of the reactor there may be coolers, filtration, washers, shift conversion, Rectisol-process, absorbents, Fischer-Tropsch-synthesis, etc.

While the invention has been described herein by way of examples in connection with what are, at present, considered to be the most preferred embodiments, it is to be understood that the invention is not limited to the disclosed embodiments, but is intended to cover various combinations or modifications of its features, and several other applications included within the scope of the invention, as defined in the appended claims. The details mentioned in connection with any embodiment above may be used in connection with another embodiment when such combination is technically feasible.

1. to 15. (canceled)

16. A method of production of synthesis gas, the method comprising the following steps:
providing raw gas that contains carbon compounds and sticky components to a reactor for processing of the raw gas by partial oxidation;
adding oxygen and steam to the raw gas stream;
reacting the oxygen and the steam with a portion of the raw gas to cause the temperature to rise to enable melting of ash and cracking of hydrocarbon compounds of the sticky components;
driving reactions occurring free of catalyst for cracking of sticky components by hydrogen of the steam to crack complex hydrocarbons into lighter volatile hydrocarbon fractions;
controlling a feed of steam to the reactor, to decompose sticky components of the raw gas and residuals accumulate as a protective slag/dust layer on an inner wall of the reactor, the reactor comprising a temperature-controlled inner wall and an outer wall, being arranged in a coaxial configuration with respect to each other, the inner wall comprising a number of water tube panel walls on a suspended support and the outer wall being a fixed wall; and
performing at least on of continuously and intermittently determining and maintaining a thickness of the protective slag/dust layer such that, when the slag/dust layer thickness has accumulated to a predetermined value, the slag/dust layer is dropped to the bottom of the reactor by rapping at least one of the inner panel walls at a time.

17. The method of according to claim 16, further comprising controlling the thickness of the protective slag/dust layer on the inner wall of the reactor by rapping the inner wall in sequence so that the temperature of the water/steam inside the inner wall is maintained within desired range.

18. The method according to claim 16, wherein the slag/dust layer is dropped to the bottom of the reactor by rapping at least one of the water tube panel walls forming the inner wall at a predetermined time interval.

19. The method according to claim 17, wherein the slag/dust layer is dropped to the bottom of the reactor by rapping at least one of the water tube panel walls forming the inner wall at a predetermined time interval.

20. The method according to claim 16, wherein, by controlling an insulating thickness of the slag/dust layer on the inner wall, the cracking of sticky components in the reactor is kept in balance.

21. The method according to claim 16, wherein alkali metal, sulphur, and chlorine components are removed from the process as attached to the slag/dust.

22. The method according to claim 16, wherein the reaction in the reactor is: C_{w}H_{z}+O₂+steam→CₓH_{y}+H₂O, where w and z are greater than x and y.

23. A reactor for processing of synthesis gas by partial oxidation from a raw gas containing carbon and sticky components, the reactor comprising:
a first intake passage for feeding the raw gas;
at least one second intake passage for controllably feeding steam and oxygen such that oxygen and steam are capable of reacting with a portion of the raw gas to cause the temperature to rise to enable melting of ash and cracking of hydrocarbon compounds of the sticky components, the reactions occurring free of catalyst for cracking of sticky components being capable of being driven by hydrogen of the steam to crack complex hydrocarbons into lighter volatile hydrocarbon fractions;
an exhaust passage for an exit of synthesis gas;
a second exhaust passage for removing at least one of separated condensed and separated solidified material;
an outer wall in a fixed configuration defining an internal space;
an inner wall assembly configured on a suspended support inside the outer wall, the inner wall being formed of a number of temperature-controlled water tube panel walls, the outer wall and the inner wall assembly being arranged in coaxial configuration in relation to each other and a longitudinal axis of the reactor; and
at least one rapping device arranged at an upper part of the inner wall for removal of a slag/dust layer on the inner wall.

24. The reactor according to claim 23, wherein the rapping device is a spring hammer.

25. The reactor according to claim 23, wherein the outer wall is temperature-controlled and is formed of at least six water tube panel walls having substantially planar cross section.

26. The reactor according to claim 24, wherein the outer wall is temperature-controlled and is formed of at least six water tube panel walls having substantially planar cross section.

27. The reactor according to claim 23, wherein the inner wall is formed of at least six water tube panel walls having a substantially planar cross section.

28. The reactor according to claim 23, wherein the inner wall has the same number of water tube panel walls as does the outer wall.

29. The reactor according to claim 23, wherein the inner wall and the outer wall are temperature controllable by way of fluid temperature inside the inner wall and the outer wall.

30. The reactor according to claim 23, wherein further comprising an enclosure that is formed as a pressure shell.

31. The reactor according to claim 23, wherein the coaxial configuration is such that a normal of the inner wall is substantially perpendicular to the longitudinal axis of the reactor.

32. The reactor according to claim 23, wherein the inner wall is shorter than the outer wall.
